(19) United States
(12) Patent Application Publication
(10) Pub. No.: US 2015/0105289 A1
(43) Pub. Date: Apr. 16, 2015

(54) BIOMARKERS FOR LOWER URINARY TRACT SYMPTOMS (LUTS)

(71) Applicant: The Regents of The University of Michigan, Ann Arbor, MI (US)

(72) Inventors: Mitchell B. Berger, Ann Arbor, MI (US); Andrew Johnston, Ann Arbor, MI (US)

(21) Appl. No.: 14/513,566

(22) Filed: Oct. 14, 2014

Related U.S. Application Data
(60) Provisional application No. 61/891,269, filed on Oct. 15, 2013.

(51) Int. Cl. G01N 33/543

(52) U.S. Cl. CPC G01N 33/54306; G01N 2800/34; G01N 2800/60; G01N 2333/485; G01N 2333/48; G01N 2333/5403; G01N 2333/5412; G01N 2333/5428; G01N 2333/5434; G01N 2333/523; G01N 2333/522; G01N 2333/70575
USPC 506/9; 435/7.92

(57) ABSTRACT

Provided herein are compositions and methods for the characterization of a subject's predisposition to developing lower urinary tract symptoms (LUTS). In particular, biomarkers are provided that identify the likelihood that a subject with develop LUTS concomitant with pelvic organ prolapse (POP), and/or the likelihood that LUTS will persist after surgical repair of POPS.

### CROSS REFERENCE TO RELATED APPLICATION

The present application claims priority to U.S. Provisional Patent Application Ser. No. 61/891,269, filed Oct. 15, 2013, the disclosure of which is herein incorporated by reference in its entirety.

### FIELD

Provided herein are compositions and methods for the characterization of a subject's predisposition to developing lower urinary tract symptoms (LUTS). In particular, biomarkers are provided that identify the likelihood that a subject with develop LUTS concomitant with pelvic organ prolapse (POP), and/or the likelihood that LUTS will persist after surgical repair of POPS.

### BACKGROUND

Women with pelvic organ prolapse (POP) often have concomitant lower urinary tract symptoms (LUTS), including urinary urgency, frequency, or incontinence. It has been documented that pelvic floor reconstructive surgery often significantly reduces these symptoms, but 40-60% of women continue to have LUTS after surgical repair.

### SUMMARY

In some embodiments, the present invention provides methods for assessing the likelihood a subject will develop lower urinary tract symptoms (LUTS) comprising detecting biomarkers in a sample. In some embodiments, LUTS comprises urinary urgency, urinary frequency, and/or urinary incontinence. In some embodiments, the level present in the sample of one or more biomarkers is indicative of likelihood of developing LUTS. In some embodiments, the level of one or more biomarkers is increased in a subject with a higher likelihood of developing LUTS. In some embodiments, the level of one or more biomarkers is decreased in a subject with a higher likelihood of developing LUTS. In some embodiments, (i) the level of one or more biomarkers is increased in a subject with a higher likelihood of developing LUTS; and (ii) the level of one or more biomarkers is decreased in a subject with a higher likelihood of developing LUTS. In some embodiments, the sample is a fluid from the subject. In some embodiments, the fluid is urine. In other embodiments, the fluid is blood, saliva, etc. In some embodiments, the subject is a human female. In some embodiments, the subject suffers from pelvic organ prolapse (POP). In some embodiments, the subject suffers from cystocele, rectocele, enterocele, sigmoidocele, urethrocele, or uterine prolapse. In some embodiments, the subject does not suffer from a form of POP. In some embodiments, the likelihood a subject will develop LUTS comprises the likelihood that a LUTS will reoccur (or persist) following treatment of the pelvic organ prolapse. In some embodiments, treatment of POP comprises surgery. In some embodiments, surgery comprises pelvic floor reconstruction. In some embodiments, biomarkers are one or more biomarkers selected from the list consisting of: heparin-binding EGF-like growth factor (hbegf), interleukin 6 (IL-6), nerve growth factor (ngf), interleukin 10 (IL-10), melanoma growth stimulating activity (Gro), soluble CD 40 ligand (SCD40L), monocyte chemoattractant protein-1 (MCP-1), interleukin 3 (IL-3), interferon gamma-induced protein 10 (IP-10), interleukin 12 (IL -12), monocyte-specific chemokine 3 (MCP 3), and macrophage inflammatory protein 1b (MIP-1b). In some embodiments, the biomarkers are one or more of HBEGF, MCP-1 and MIP-1b. In some embodiments, the biomarkers are detected by measuring protein levels. In some embodiments, the biomarkers are assessed by measuring expression levels, mRNA levels, etc. In some embodiments, the level of the biomarkers are differentially weighted to determine the likelihood of developing LUTS.

In some embodiments, the present invention provides methods of determining a treatment course for a subject suffering from pelvic organ prolapse (POP) with concomitant lower urinary tract symptoms (LUTS): (a) assessing the likelihood that LUTS will persist after surgical treatment of POP based on the level of one or more biomarkers in a sample from the subject; (b) selecting a suitable treatment course for the subject, wherein: (i) surgery alone is indicated if the likelihood that LUTS will persist after surgical treatment of POP is low; and (ii) medical treatment for LUTS with or without surgery for POP is indicated if the likelihood that LUTS will persist after surgical treatment of POP is high. In some embodiments, the level of one or more biomarkers in a sample is assessed by providing a testing lab with a sample from the subject and receiving results of a test for the biomarkers from the testing lab. In some embodiments, the results comprise the level of each of said biomarkers. In some embodiments, the results comprise a risk profile calculated based on the level of said biomarkers. In some embodiments, methods further comprise obtaining a sample from the subject. In some embodiments, methods further comprise implementing the selected treatment course.

In some embodiments, the present invention provides methods for assessing the likelihood that the subject from which a sample was obtained will develop LUTS, comprising: (a) receiving the sample obtained from the subject; (b) quantitating the levels of a plurality of biomarkers indicative of the likelihood that a subject will develop LUTS; and (c) generating a LUTS risk profile based on the levels of a plurality of biomarkers. In some embodiments, a computer-based algorithm is used to convert the levels of a plurality of biomarkers into the risk profile. In some embodiments, the level of the biomarkers are differentially weighted to determine the LUTS risk profile. In some embodiments, the risk profile is a quantitative value or a qualitative risk. In some embodiments, methods further comprise: (d) generating a report indicating the likelihood of developing LUTS of the subject from which the sample was obtained. In some embodiments, the likelihood that the subject from which a sample was obtained will develop LUTS comprises the likelihood that LUTS will persist after surgical treatment of POP.

In some embodiments, the present invention provides kits for assessing the likelihood of LUTS comprising reagents for detecting the level of a plurality of biomarkers. In some embodiments, the biomarkers are one or more biomarkers selected from the list consisting of: hbegf, IL-6, ngf, IL-10, Gro, SCD40L, MCP-1, IL-3, IP-10, IL -12, MCP 3, and MIP-1b. In some embodiments, the biomarkers are one or more of HBEGF, MCP-1 and MIP-1b. In some embodiments, a kit consists of, or consists essentially of, reagents for detecting biomarkers for assessing the likelihood of LUTS. In some embodiments, a kit consists of, or consists essentially of, reagents for detecting 2 or more biomarkers, 3 or more biomarkers, 4 or more biomarkers, 5 or more biomarkers . . . 10 or more biomarkers . . . 15 or more biomarkers . . . 20 or more biomarkers . . . 25 or more biomarkers . . . 30 or more biomarkers . . . 35 or more biomarkers . . . 40 or more biomarkers, etc. In some embodiments, a kit consists of, or consists essentially of, reagents for detecting fewer than 500 biomarkers . . . fewer than 400 biomarkers . . . fewer than 300 biomarkers . . . fewer than 200 biomarkers . . . fewer than 100 biomarkers . . . fewer than 75 biomarkers . . . fewer than 50 biomarkers . . . fewer than 40 biomarkers . . . fewer than 30 biomarkers . . . fewer than 20 biomarkers . . . fewer than 10 biomarkers, etc. In some embodiments, a kit does not provide reagents for detecting markers not related to assessing the likelihood of LUTS. In some embodiments, reagents comprise antibodies. In other embodiments, reagents comprise probes, fluorescent labels, radiolables, primers, etc. In some embodiments, kits further comprise buffers or other reagents to enable biomarker detection in urine.

In some embodiments, provided herein are kits for conducting assays to identify the expression of the markers. In some such embodiments, the kits comprise reagents (e.g., antibodies, probes, primers, buffers, etc.) and other components (e.g., software, instructions, data sets) necessary, sufficient, or useful for conducting any of the methods described herein. In some embodiments, the kits provide reagents in multiplex format for the simultaneous analysis of multiple markers (e.g., on one reaction mixture, container, or devices (e.g., multi-well card or plate)). In some embodiments, the kits comprise, consist, or consist essentially of the reagents needed to assess the plurality of markers to provide a desired diagnostic result. In some such embodiments, for example, for cost-efficiency, such kits do not include reagents (e.g., primers and probes) for analyzing other markers (e.g., rather than using a gene chip to assess expression of all expression markers, the kit only detects the specific markers needed to make the diagnostic assessment).

Also provided herein are reaction mixtures comprising sample (e.g., urine or sample derived therefrom) and reagents (e.g., from any of the above kits or methods) for assessing level of the biomarkers described herein (e.g., alone or in combination with other biomarkers not listed herein). In some embodiments, the reaction mixtures are generated by conducting a method as described herein.

In some embodiments, a software or hardware component receives the results of multiple assays and/or markers and determines a single value result to report to a user that indicates a conclusion (e.g., high risk of LUTS, low risk or LUTS, high likelihood of LUTS risk persisting after surgery to repair pelvic floor, low likelihood of LUTS risk persisting after surgery to repair pelvic floor, etc.). Related embodiments calculate a risk factor based on a mathematical combination (e.g., a weighted combination, a linear combination) of the results from multiple assays and/or markers.

Some embodiments comprise a storage medium and memory components. Memory components (e.g., volatile and/or nonvolatile memory) find use in storing instructions (e.g., an embodiment of a process as provided herein) and/or data. Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.). In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship.

In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

### DEFINITIONS

To facilitate an understanding of the present technology, a number of terms and phrases are defined below. Additional definitions are set forth throughout the detailed description. Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

“Amplification” is a special case of nucleic acid replication involving template specificity. It is to be contrasted with non-specific template replication (e.g., replication that is template-dependent but not dependent on a specific template). Template specificity is here distinguished from fidelity of replication (e.g., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Amplification of nucleic acids generally refers to the production of multiple copies of a polynucleotide, or a portion of the polynucleotide, typically starting from a small amount of the polynucleotide (e.g., a single polynucleotide molecule, 10 to 100 copies of a polynucleotide molecule, which may or may not be exactly the same), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), Hot-start PCR (see, e.g., U.S. Pat. Nos. 5,773,258 and 5,338,671; each herein incorporated by reference in their entireties), intersequence-specfic PCR, inverse PCR (see, e.g., Triglia, et al et al. (1988) Nucleic Acids Res., 16:8186; herein incorporated by reference in its entirety), ligation-mediated PCR (see, e.g., Guilfoyle, R. et al et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93 (13) 9821-9826; herein incorporated by reference in its entirety), miniprimer PCR, multiplex ligation-dependent probe amplification (see, e.g., Schouten, et al., (2002) Nucleic Acids Research 30 (12): e57; herein incorporated by reference in its entirety), multiplex PCR (see, e.g., Chamberlain, et al., (1988) Nucleic Acids Research 16 (23) 11141-11156; Ballabio, et al., (1990) Human Genetics 84 (6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16 (15) 7351-7367; herein incorporated by reference in its entirety), real time PCR (see, e.g., Higuchi, et al et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19 (14) 4008; Roux, K. (1994) Biotechniques 16 (5) 812-814; Hecker, et al., (1996) Biotechniques 20 (3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

As used herein, the term “nucleic acid detection assay” refers to any method of determining the nucleotide composition of a nucleic acid of interest. Nucleic acid detection assay include but are not limited to, DNA sequencing methods, probe hybridization methods, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Barnay Proc. Natl. Acad. Sci USA 88, 189-93 (1991)); and sandwich hybridization methods (e.g., U.S. Pat. No. 5,288,609, herein incorporated by reference in its entirety).

The term “amplifiable nucleic acid” refers to a nucleic acid that may be amplified by any amplification method. It is contemplated that “amplifiable nucleic acid” will usually comprise “sample template.”

The term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (e.g., in the presence of nucleotides and an inducing agent such as a DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject developing LUTS, the likelihood LUTS will persist following treatment of POP (e.g., surgical repair), or the likelihood that such a subject will respond favorably to a compound (e.g., a pharmaceutical, e.g., a drug) or other treatment for LUTS.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, by be combined with other nucleic acids or molecules. For example, an isolated nucleic acid may be present in a host cell in which into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to an animal cell or tissue. In another sense, it is meant to include a specimen or culture obtained from any source, as well as other biological samples. Biological samples may be obtained from plants or animals (including humans) and encompass fluids (e.g., urine, blood, etc.), solids, tissues, and gases. These examples are not to be construed as limiting the sample types applicable to the present invention.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. In typical embodiments, a subject is a female.

As used herein, the term “pelvic organ prolapse” refers to a condition in which organs of the lower abdomen (e.g., uterus, bladder) slip out of a healthy position (e.g., fall down), typically into or through the vagina. Pelvic organ prolapse may refer to any or all of “cystocele” (e.g., herniation of the bladder though the pubovesical fascia), “rectocele” (e.g., prolapse of rectal tissue through the rectovaginal septum), “enterocele” (e.g., protrusion of the small intestines and/or peritoneum), “sigmoidocele” (e.g., descending of the sigmoid colon into the lower pelvic cavity), “urethrocele” (e.g., prolapse of the female urethra into the vagina), “uterine prolapse” (e.g., sliding or falling of the uterus into or through the vaginal canal), etc.

### DETAILED DESCRIPTION

Provided herein are compositions and methods for the characterization of a subject's predisposition to developing lower urinary tract symptoms (LUTS). In particular, biomarkers are provided that identify the likelihood that a subject with develop LUTS concomitant with pelvic organ prolapse (POP), and/or the likelihood that LUTS will persist after surgical repair of POPS.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. Although the disclosure herein refers to certain embodiments, it is to be understood that these embodiments are presented by way of example and not by way of limitation.

In some embodiments, the technology relates to assessing the level (e.g., concentration) of combinations of biomarkers comprising consisting essentially of, or consisting of, e.g., 2, 3, 4, 5, 6, 7, 8, 9, 10, 11, 12, 13, 14, 15, 16, 17, 18, 19, 20, 21, 22, 23, 24, 25, 26, 27, 27, 29, 30, or more biomarkers (e.g., including one or more of hbegf, IL-6, ngf, IL-10, Gro, SCD40L, MCP-1, IL-3, IP-10, IL-12, MCP 3, and MIP-1b). In some embodiments, a panel or multiplex assay or multiple assays are conducted that assess the level of two or more markers selected from hbegf, IL-6, ngf, IL-10, Gro, SCD40L, MCP-1, IL-3, IP-10, IL-12, MCP 3, and MIP-1b. The quantification results are analyzed to generate a risk score (e.g., via computer algorithm weighing each of the markers' concentration (e.g., in urine), and, for example, comparing to a look-up table of established risk associated with such biomarker level; in some embodiments, sub-categorized by patient sub-type (e.g., based on age, disease type (e.g., POP), or other desired factor)).

In some embodiments, assessing the level of more than one biomarker increases the specificity and/or sensitivity of a screen or diagnostic. In some embodiments, a biomarker or a combination of biomarkers discriminates between subjects likely responsive or unresponsive to a particular therapy (e.g., surgery). Patient responses are predicted by various combinations of biomarkers, e.g., as identified by statistical techniques. The technology provides methods for identifying predictive combinations and validated predictive combinations of biomarkers.

Some embodiments comprise detection of nucleic acids of expression thereof. Nucleic acid expression may be assessed by any desired technique. In some embodiments, nucleic acid (e.g., RNA) is first isolated from a sample. Nucleic acid may be isolated by any means, including the use of commercially available kits. Briefly, wherein the nucleic acid of interest is encapsulated in by a cellular membrane the biological sample may be disrupted and lysed by enzymatic, chemical or mechanical means. The nucleic acid is then recovered from the solution. This may be carried out by means of a variety of methods including salting out, organic extraction, or binding of the nucleic acid to a solid phase support. The choice of method will be affected by several factors including time, expense, and required quantity and/or quality of nucleic acid desired. All clinical sample types are suitable for use in the present method, e.g., cell lines, histological slides, biopsies, paraffin-embedded tissue, body fluids, stool, colonic effluent, urine, blood plasma, blood serum, whole blood, isolated blood cells, cells isolated from the blood, and combinations thereof.

In some embodiments, the technology relates to a method for treating a patient (e.g., a patient with POP), the method comprising determining the level of one or more biomarkers as provided herein and administering a treatment to the patient based on the results of analysis. The treatment may be surgical (alone or in combination with other therapies) or medical, including, but not limited to, use of a pharmaceutical compound, a vaccine physical therapy, etc. The biomarkers may also be used to monitor a patient during a course of therapy to determine, for example, whether the therapy is or remains or become efficacious and to determine whether changes in therapy should be made.

The biomarkers described herein also find use in research applications for the study of LUTS, POP, etc. In some embodiments, cells expression the biomarkers described herein are provided. In some embodiments, cells with reduced expression (e.g., by siRNA, by knockout, by mutation), are provided. In some embodiments, transgenic animals under- or over-expression the biomarkers described herein are provided.

### EXPERIMENTAL

### Example 1

### Case-Control Study

Women with cystocele often have lower urinary tract symptoms (LUTS), and LUTS frequently resolve after prolapse reduction. Experiments were conducted during development of embodiments of the present invention to compare levels of urinary biomarkers in (1) women with and without cystocele, and (2) women whose LUTS do or do not improve after prolapse repair. A case-control study of women with cystocele (Ba≧+1) (cases) and controls with normal support undergoing benign gynecologic surgery was performed. Baseline demographics were recorded. All subjects completed the MESA and PFDI-20 questionnaires preoperatively. Intraop urine specimens were obtained, spun, and the supernatants stored at −80° C. Cases repeated the surveys at 6 weeks postop. Urinary biomarkers were assayed using ELISA (creatinine, NGF and HB-EGF) or Milliplex assay (sCD40L, IL-3, IL-6, IL-10, IL-12, MCP-1, MCP-3, GRO, IP-10, MIP-1α, and MIP-1β). All biomarker concentrations were normalized to creatinine concentration.

Demographics of the 93 cases and 61 controls are presented in Table 1. More severe LUTS (higher scores on the Urinary Distress Inventory-6 (UDI-6) or Medical, Epidemiological and Social Aspects of Aging (MESA) questionnaires) were reported by cases than controls (Table 2). Cases had significantly higher urinary levels of MCP-1 than the controls (Table 2).

TABLE 1
Demographics

| Demographic | Controls (n = 61) | | Cases (n = 93) | | p value |
|---|---|---|---|---|---|
| Age (years) | 48.0 ± 13.4 | | 63.3 ± 10.3 | | <0.001 |
| BMI (kg/m²) | 29.9 ± 7.7 | | 27.8 ± 5.7 | | 0.06 |
| Parity | 2 | (0, 6) | 2 | (0, 11) | <0.001 |
| Post-Void Residual (mL) | 37.8 ± 43.4 | | 56.1 ± 53.0 | | 0.20 |
| Point Ba (cm) | −2 | (−3, −1) | 3 | (1, 9) | <0.001 |

TABLE 2
LUTS

| | Controls | Cases | p value |
|---|---|---|---|
| UDI-6 Score | 24.7 ± 20.7 | 37.3 ± 25.7 | 0.002 |
| Total MESA Score | 9.1 ± 8.5 | 13.6 ± 9.7 | 0.003 |
| MESA Stress Subscale Score | 6.5 ± 5.6 | 8.6 ± 6.9 | 0.04 |
| MESA Urge Subscale Score | 2.7 ± 3.4 | 5.0 ± 4.0 | <0.001 |
| [MCP-1] (pg/mL) | 2.3 (0.2, 7.3) | 3.1 (0.2, 11.3) | 0.02 |

Postop changes in LUTS were assessed: responders were defined as subjects whose (1) UDI score improved ≧11 points, (2) total MESA score improved ≧40%, (3) MESA stress subscale score improved ≧42%, or (4) MESA urge subscale score improved ≧40%. Demographics were similar in responders and non-responders for all surveys except the MESA urge subscale, for which responders had lower BMI and higher parity. Urinary biomarker levels were also compared. Responders (UDI) had higher concentrations of IL-6, IL-10, and MIP-1β than non-responders. Responders (total MESA and MESA stress subscale scores) had significantly lower NGF concentrations than non-responders. No significant biomarker differences were seen for the MESA urge subscale.

Experiments conducted during development of embodiments of the present invention demonstrated that urinary MCP-1, an inflammatory cytokine elevated in urine from patients with OAB, is higher in women with cystocele than normal support. Further, urinary biomarkers associated with postop improvement of LUTS include higher levels of other inflammatory markers (IL-6, IL-10 & MIP-1β) and lower levels of NGF, a marker of OAB.

### Example 2

### Biomarker Analysis

Standard logistic regression was performed using the response variables on clinical characteristics (e.g., age, body mass index (BMI), parity, and point B anterior landmark Ba all entered as continuous variables) and the area under the curve (predictive power) was assessed with five-fold cross validation. Elastic net logistic regression was used, including all clinical characteristics (forced in the model) and 13 biomarkers. This method provides good classification performance while choosing a minimal number of predictor variables (e.g., employs a trade-off between the lasso and ridge regression penalties). Experiments were performed with alpha ranging from 0.1 to 1 and the parameter λ was tuned by five-fold cross validation. The area under the ROC in the models from elastic net regression is that from the five-fold cross validation.

For the outcome of any response, 5 biomarkers were retained: hbegf, IL-10, MCP-1, IL-12, and MIP-1b for a five-fold cross validation area under ROC of 0.769. For the outcome of response to MESA, 8 markers were retained: hbegf, IL-6, MCP-1, Gro, IL-12, IL-3, MCP 3, and MIP-1b for a five-fold CV area under ROC of 0.682. For the outcome of response to MESA stress, 12 markers were retained: ngf, hbegf, SCD40L, IL-6, MCP-1, Gro, IL-12, IL-3, IP-10, MCP 3, and MIP-1b for a five-fold CV area under ROC of 0.79. For the outcome of response to MESA urge, 3 markers were retained: hbegf, MCP-1, and MIP-1b for a five-fold CV area under ROC of 0.72. For the outcome of response to UDI, 12 markers were retained: ngf, hbegf, SCD40L, IL-6, MCP-1, Gro, IL-12, IL-3, IP-10, MCP 3, and MIP-1b for a five-fold CV area under ROC of 0.80. The analysis indicates that HBEGF, MCP-1 and MIP-1b may have the strongest predictive value of the biomarkers.

TABLE 3
Association of any response with clinical characteristics

| Variable | Estimate | AUC |
|---|---|---|
| Intercept | 3.257 | 0.704 |
| Age | −0.028 | |
| BMI | −0.076 | |
| Parity | 0.136 | |
| BA | 0.498 | |

TABLE 4
Association of any response with clinical characteristics & biomarkers
from Elastic Net Regression
Alpha = 0.9, lambda = 0.02072

| Variable | Estimate | AUC |
|---|---|---|
| Intercept | 1.915 | 0.769 |
| Age | −0.023 | |
| BMI | −0.151 | |
| Parity | 0.295 | |
| BA | 0.585 | |
| HBEGF | 0.013 | |
| IL-10 | 0.783 | |
| MCP1 | 0.002 | |
| IL-12 | 2.187 | |
| MIP-1b | 0.220 | |

TABLE 5
MESA, Association of any response with clinical characteristics

| Variable | Estimate | AUC |
|---|---|---|
| Intercept | −1.522 | 0.579 |
| Age | −0.009 | |
| BMI | −0.062 | |
| Parity | 0.229 | |
| BA | 0.228 | |

TABLE 6
MESA, Association of any response with clinical characteristics &
biomarkers from Elastic Net Regression
Alpha = 0.15, lambda = 0.123004

| Variable | Estimate | AUC |
|---|---|---|
| Intercept | 0.610 | 0.682 |
| Age | −0.003 | |
| BMI | −0.073 | |
| Parity | 0.260 | |
| BA | 0.210 | |
| HBEGF | 0.004 | |
| IL-6 | 0.061 | |
| MCP1 | 0.00006 | |
| GRO | 0.0003 | |
| IL-12 | 0.709 | |
| IL-3 | 0.442 | |
| MCP 3 | 0.091 | |
| MIP-1b | 0.046 | |

TABLE 7
MESA STRESS, Association of any response with clinical characteristics

| Variable | Estimate | AUC |
|---|---|---|
| Intercept | 1.822 | 0.559 |
| Age | −0.009 | |
| BMI | −0.078 | |
| Parity | 0.129 | |
| BA | 0.371 | |

TABLE 8
MESA STRESS, Association of any response with clinical
characteristics & biomarkers from Elastic Net Regression
Alpha = 1, lambda = 0.00007583

| Variable | Estimate | AUC |
|---|---|---|
| Intercept | −1.904 | 0.793 |
| Age | 0.094 | |
| BMI | −0.231 | |
| Parity | 0.601 | |
| BA | −0.221 | |
| NGF | −0.040 | |
| HBEGF | 0.021 | |
| SCD 40-L | −0.671 | |
| IL-10 | 9.845 | |
| IL-6 | 4.903 | |
| MCP1 | −0.0008 | |
| GRO | 0.420 | |
| IL-12 | 1.518 | |
| IL-3 | 6.503 | |
| IP-10 | −0.027 | |
| MCP 3 | 4.846 | |
| MIP-1b | 0.3777 | |

TABLE 9
MESA Urge, Association of any response with clinical characteristics

| Variable | Estimate | AUC |
|---|---|---|
| Intercept | −1.966 | 0.645 |
| Age | 0.041 | |
| BMI | −0.095 | |
| Parity | 0.577 | |
| BA | 0.225 | |

TABLE 10
MESA Urge, Association of any response with clinical characteristics &
biomarkers from Elastic Net Regression
Alpha = .9, lambda = 0.0535

| Variable | Estimate | AUC |
|---|---|---|
| Intercept | −2.774 | 0.724 |
| Age | 0.041 | |
| BMI | −0.109 | |
| Parity | 0.544 | |
| BA | 0.164 | |
| HBEGF | 0.013 | |
| MCP1 | 0.0002 | |
| MIP-1b | 0.053 | |

TABLE 11
UDI, Association of any response with clinical characteristics

| Variable | Estimate | AUC |
|---|---|---|
| Intercept | −0.397 | 0.585 |
| Age | −0.007 | |
| BMI | 0.013 | |
| Parity | 0.059 | |
| BA | 0.291 | |

TABLE 12
UDI, Association of any response with clinical characteristics & biomarkers
from Elastic Net Regression
Alpha = 1, lambda = .00001145

| Variable | Estimate | AUC |
|---|---|---|
| Intercept | 8.437 | 0.801 |
| Age | 0.054 | |
| BMI | −0.396 | |
| Parity | 0.089 | |
| BA | 0.776 | |
| NGF | −0.024 | |
| HBEGF | −0.009 | |
| SCD 40-L | −1.056 | |
| IL-10 | 13.830 | |
| IL-6 | 9.392 | |
| MCP1 | −0.014 | |
| GRO | −0.033 | |
| IL-12 | 8.568 | |
| IL-3 | −17.835 | |
| IP-10 | 0.1000 | |
| MCP 3 | 0.889 | |
| MIP-1b | 0.573 | |

We claim:

1. A method for assessing the likelihood a human female subject will develop LUTS comprising detecting the level of one or more biomarkers in a sample, wherein the level of the one or more biomarkers is indicative of likelihood of developing LUTS, and wherein the biomarkers are one or more biomarkers selected from the list consisting of: hbegf, IL-6, ngf, IL-10, Gro, SCD40L, MCP-1, IL-3, IP-10, IL-12, MCP 3, and MIP-1b.

2-4. (canceled)

5. The method of claim 1, wherein:
(i) the level of one or more biomarkers is increased in a subject with a higher likelihood of developing LUTS; and/or
(ii) the level of one or more biomarkers is decreased in a subject with a higher likelihood of developing LUTS.

6-8. (canceled)

9. The method of claim 8, wherein the subject suffers from pelvic organ prolapse (POP).

10. The method of claim 9, wherein the subject suffers from cystocele, rectocele, enterocele, sigmoidocele, urethrocele, or uterine prolapse.

11. (canceled)

12. The method of claim 11, wherein said treatment comprises surgery.

13. The method of claim 12, wherein said surgery comprises pelvic floor reconstruction.

14. (canceled)

15. The method of claim 1, wherein the biomarkers are one or more of HBEGF, MCP-1 and MIP-1b.

16. The method of claim 1, wherein biomarkers are detected by measuring protein levels.

17. The method of claim 1, wherein the level of the biomarkers are differentially weighted to determine the likelihood of developing LUTS

18. The method of claim 1, wherein LUTS comprises urinary urgency, urinary frequency, and/or urinary incontinence.

19. A method of determining a treatment course for a subject suffering from pelvic organ prolapse (POP) with concomitant lower urinary tract symptoms (LUTS):
(a) assessing the likelihood that LUTS will persist after surgical treatment of POP based on the level of one or more biomarkers in a sample from the subject;
(b) selecting a suitable treatment course for the subject, wherein:
(i) surgery alone is indicated if the likelihood that LUTS will persist after surgical treatment of POP is low; and
(ii) medical treatment for LUTS with or without surgery for POP is indicated if the likelihood that LUTS will persist after surgical treatment of POP is high.

20. The method of claim 19, wherein the level of one or more biomarkers in a sample is assessed by providing a testing lab with a sample from the subject and receiving results of a test for the biomarkers from the testing lab.

21. (canceled)

22. The method of claim 20, wherein the results comprise a risk profile calculated based on the level of said biomarkers.

23. The method of claim 19, further comprising obtaining a sample from the subject.

24. The method of claim 19, further comprising implementing the selected treatment course.

25. A method for assessing the likelihood that the subject from which a sample was obtained will develop LUTS, comprising:
(a) receiving the sample obtained from the subject;
(b) quantitating the levels of a plurality of biomarkers indicative of the likelihood that a subject will develop LUTS; and
(c) generating a LUTS risk profile based on the levels of a plurality of biomarkers.

26. (canceled)

27. The method of claim 26, wherein the level of the biomarkers are differentially weighted to determine the LUTS risk profile.

28. (canceled)

29. The method of claim 25, further comprising:
(d) generating a report indicating the likelihood of developing LUTS of the subject from which the sample was obtained.

30. The method of claim 25, wherein the likelihood that the subject from which a sample was obtained will develop LUTS comprises the likelihood that LUTS will persist after surgical treatment of POP.

31. A kit for assessing the likelihood of LUTS comprising reagents for detecting the level of a plurality of biomarkers selected from the list consisting of: hbegf, IL-6, ngf, IL-10, Gro, SCD40L, MCP-1, IL-3, IP-10, IL-12, MCP 3, and MIP-1b.

32-35. (canceled)
